(19) United States
(12) Patent Application Publication
(10) Pub. No.: US 2019/0282395 A1
(43) Pub. Date: Sep. 19, 2019

(54) Thermal Pack

(71) Applicant: Oztent Australia Pty. Limited, Wetherill Park (AU)

(72) Inventor: Peter Long, Wetherill Park (AU)

(21) Appl. No.: 16/355,093

(22) Filed: Mar. 15, 2019

(30) Foreign Application Priority Data
Mar. 16, 2018 (AU) 2018100334

(51) Int. Cl. A61F 7/02

(52) U.S. Cl. CPC A61F 7/02; A61F 2007/0255; A61F 2007/023

(57) ABSTRACT

A thermal pack having a pouch, the pouch having a first wall and a second wall coupled to the first wall to form an interior volume. The interior volume configured to retain a thermal means. A thermal resistivity of the first wall is greater than a thermal resistivity of the second wall such that heat flow into, or out of, the interior volume is primarily through the second wall.

### FIELD

The present invention relates to a thermal pack.

### BACKGROUND

Thermal packs for providing heating and/or cooling to a person are known. Known heating packs typically radiate heat uniformly in all directions. This results in a significant amount of heat being directed away from the person using the heating pack.

Similarly, known cooling packs typically absorb heat uniformly from all directions. This results in the cooling pack absorbing a significant amount of heat from its surroundings rather than from the person using the cooling pack.

In view of the above, it will be appreciated that known thermal packs are inefficient at providing targeted heating or cooling directed to a part of the person using the thermal pack.

Further, thermal packs traditionally require the user to hold the pack to a part of the body where heating or cooling is to be applied. This results in an inefficient and sometimes impractical application of the product particularly when activity or movement of the person using the thermal pack is involved or if they are using a variety of different types of furniture or structures.

### OBJECT OF THE INVENTION

It is an object of the present invention to overcome or at least ameliorate one or more of the above disadvantages.

### SUMMARY OF INVENTION

The present invention provides a thermal pack having a pouch, the pouch comprising:

a first wall; and

a second wall coupled to the first wall to define an interior volume, the interior volume configured to retain a thermal means in use,

wherein a thermal resistivity of the first wall is greater than a thermal resistivity of the second wall such that heat flow into, or out of, the interior volume is primarily through the second wall.

In an embodiment, a surface of the first wall comprises a heat reflective material.

In an embodiment, the heat reflective material at least partially defines the interior volume.

In an embodiment, the interior volume is configured to removably receive the thermal means.

In an embodiment, the temperature pack further comprises a means configured to removably attach the temperature pack to a person or a piece of furniture.

### BRIEF DESCRIPTION OF DRAWINGS

A preferred embodiment of the invention will be described hereinafter, by way of example only, with reference to the accompanying drawings, wherein:

FIG. 1 is a perspective view of the thermal pack according to an embodiment of the present invention;

FIG. 2 is a top view of the thermal pack of FIG. 1;

FIG. 3 is a front view of the thermal pack of FIG. 1; and

FIG. 4 is an example of a thermal means that may be received in the interior volume of the thermal pack of FIG. 1;

FIG. 5 is a perspective view of a sleeping bag according to another embodiment of the present invention;

FIG. 6 is a partial end view of the sleeping bag of FIG. 5;

FIG. 7 is a partial view of one of the thermal pouches of the sleeping bag of FIG. 5; and

FIG. 8 is a partial side view of the sleeping bag of FIG. 5.

### DESCRIPTION OF EMBODIMENTS

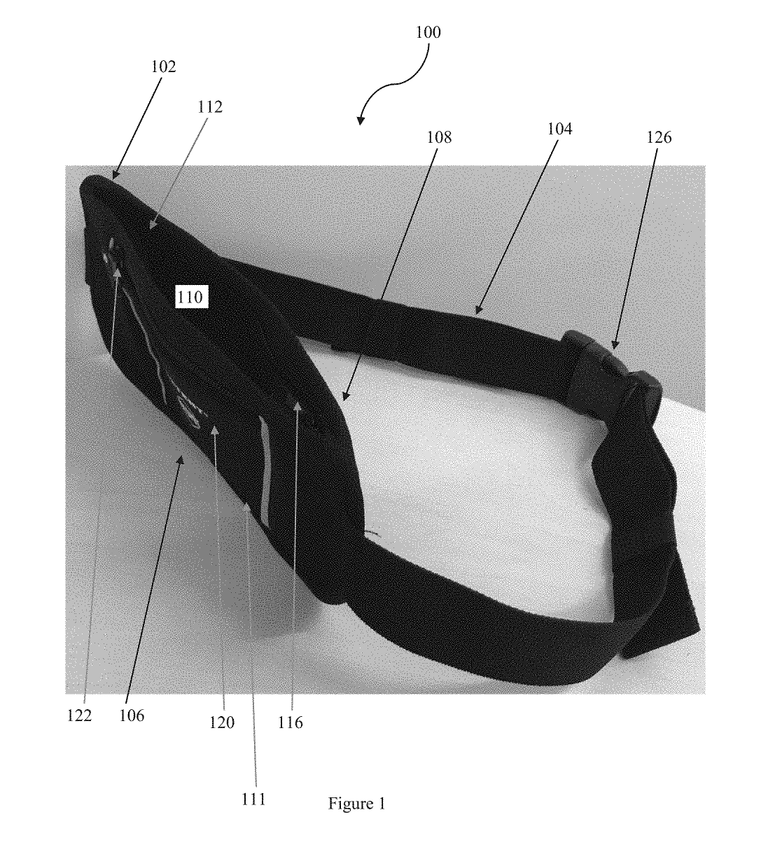

FIG. 1 shows a thermal pack 100 according to an embodiment of the present invention. The thermal pack 100 has a pouch 102 and a strap 104 coupled to the pouch 102. The strap 104 allows the thermal pack 100 to be removably attached to a person or a piece of furniture such as, for example, a camping chair. The coupling method can be any typical method such as stitching, press studs, hook and loop fasteners, adhesives or the like. The strap can also be of any length and include adjustment means, such as clips or the like.

The pouch 102 has a first wall 106 and a second wall 108. The first wall 106 has a thermal resistivity that is greater than a thermal resistivity of the second wall 106. It is envisaged that the first wall 106 is at least partially formed of a neoprene material and that the second wall 108 is formed of a synthetic fabric material (e.g. polyester). It will be appreciated that the first wall 106 and the second wall 108 can be formed by any other suitable materials known in the art so long as the thermal resistivity of the first wall 106 is greater than the thermal resistivity of the second wall 108.

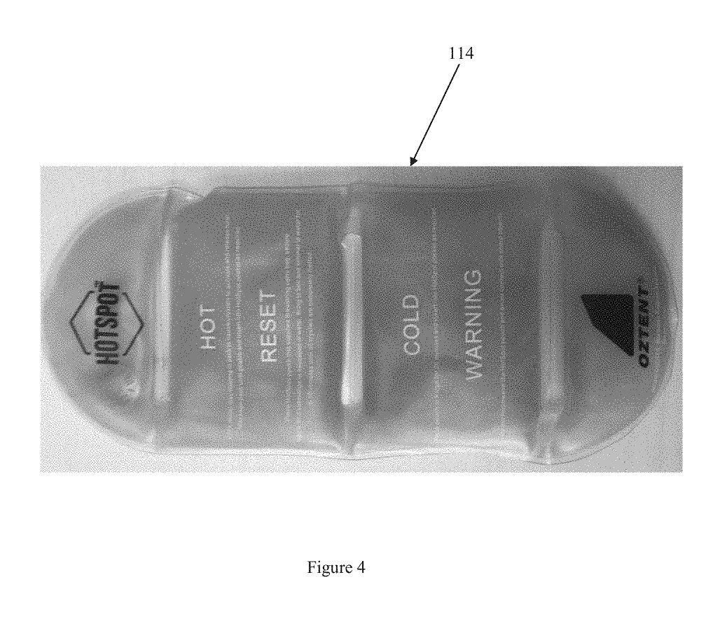

The second wall 108 is coupled to the first wall 106 to define an interior volume 110 having a closed end 111 and an opening 112. The interior volume 110 is configured to retain a thermal means 114 (see FIG. 4) therein. As the thermal resistivity of the first wall 106 is greater than the thermal resistivity of the second wall 108, it will be appreciated that heat flow into, or out of, the interior volume 110 will be primarily through the second wall 108.

The opening 112 in one embodiment has a zip 116 for opening and closing the opening 112. It will be appreciated that opening the opening 112 with the zip 116 allows a thermal means 114 to be received and removed from the interior volume 110, and that closing the opening 112 with the zip 116 allows a thermal means 114 to be retained in the interior volume 110. It is envisaged that any other suitable means known in the art may be used to open and close the opening 112 such as, for example, hook and loop fasteners, magnetic means, buttons, press studs or the like.

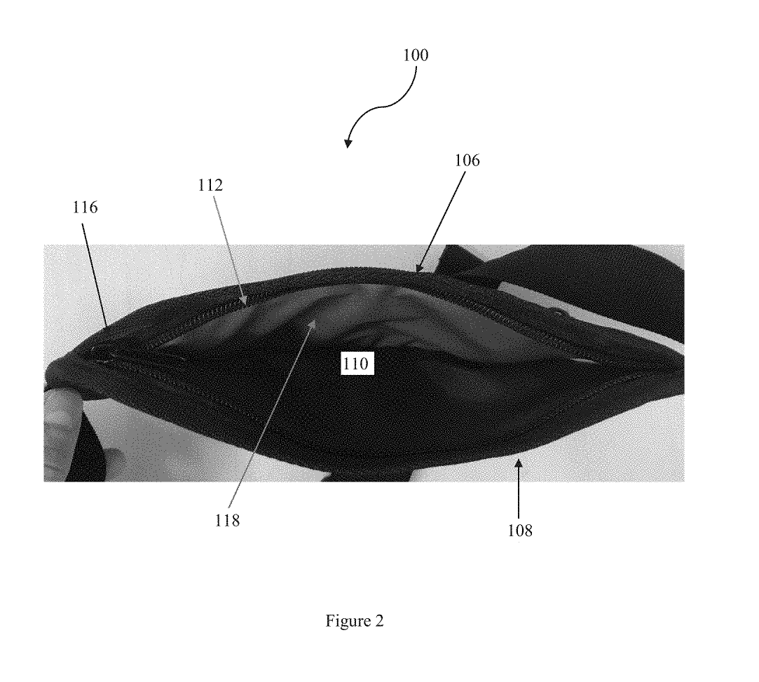

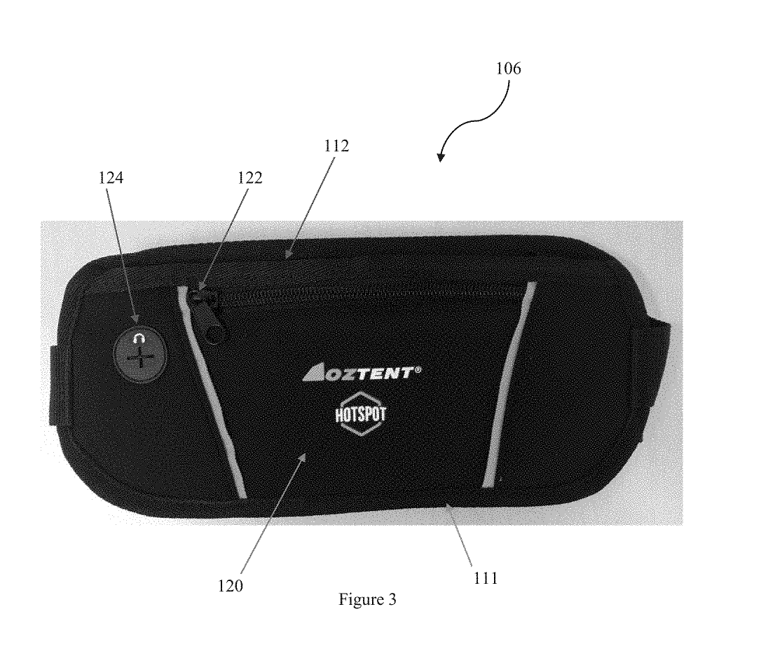

Referring to FIG. 2, the first wall 106 has a reflective material 118 that at least partially defines the interior volume 110. Referring to FIG. 3, the first wall 106 also has a pocket 120 that can be opened and closed with a zip 122. The pocket 120 has an opening 124 that allows the cable of, for example, earphones to pass from a mobile phone, or the like, retained in the pocket 120 to a person using the temperature pack 100. It is envisaged that any other suitable means known in the art may be used to open and close the pocket 120 such as, for example, discussed above.

Referring to FIG. 1, the strap 104 has a clip 126 that allows the thermal pack 100 to be removably attached to a person, a piece of furniture or the like. The clip 126 also allows the length of the strap 104 to be adjusted such that the thermal pack 100 can be fitted to different body parts of a person, people of different sizes, and a variety of different types of furniture or structures.

Use of the thermal pack 100 for providing heat to a person will now be described. A thermal means 114 configured to radiate heat (e.g. a heat pack or a hot/cold pack radiating heat) is received in the interior volume 110 and the opening 112 closed using the zip 116. The thermal pack 100 is then attached to the body of a person, for example, using the strap 104 such that the second wall 108 is adjacent, or in contact with, the body of a user. The length of the strap 104 is adjusted using the clip 126 so as to retain the thermal pack 100 in the desired position relative to the user's body. Alternatively, the thermal pack 100 can be attached to a piece of furniture such as, for example, a camping chair using the strap 104 such that the second wall 108 will be adjacent, or in close proximity, to the body of a person using the piece of furniture. For example, to heat a person's back.

Heat radiating from the thermal means 114 will primarily flow out of the interior volume 110 through the second wall 108, given that the thermal resistivity of the first wall 106 is greater than the thermal resistivity of the second wall 108. Further, the heat reflective material 118 will reflect heat dissipated from the thermal means 114 towards the second wall 108, thereby decreasing the amount of heat flowing out of the interior volume 110 through the first wall 106 and increasing the amount of heat flowing out of the interior volume 110 through the second wall 108.

As the second wall 108 is positioned adjacent, or in contact, with the body of a user, a majority of the heat radiating from the thermal means 114 will pass through the second wall 108 to the user. It will be appreciated that more heat will pass from the interior volume 110 to the user compared to the amount of heat dissipated from the interior volume 110 through the first wall 106 to the surroundings. As less heat is dissipated to the surroundings, more heat can be provided to the user before the thermal means 114 reaches thermal equilibrium.

Use of the thermal pack 100 for providing cooling to a person will now be described. A thermal means 114 configured to absorb heat (e.g. a cool pack or a hot/cold pack absorbing heat) is received in the interior volume 110 and the opening 112 closed using the zip 116. The thermal pack 100 is then attached to a person or a piece of furniture in the same way described above in paragraph [0026].

It will be appreciated that more heat will flow into the interior volume 110, and be absorbed by the thermal means 114, through the second wall 108 compared to the first wall 106, given the first wall 106 has a greater thermal resistivity than the thermal resistivity of the second wall 108. Further, the heat reflective material 118 will reflect heat flowing through the first wall 106 towards the interior volume 110, thereby reducing the amount of heat flowing into the interior volume 110 through the first wall 106.

As the second wall 108 is adjacent, or in contact with, the user's body, the thermal means 114 will absorb the user's body heat through the second wall 108. Accordingly, it will be appreciated that the thermal means 114 will absorb less heat from the surroundings through the first wall 106 and more body heat from the user through the second wall 108. As less heat is absorbed from the surroundings, more body heat can be absorbed from the user before the thermal means 114 reaches thermal equilibrium.

Although it has been described above that the thermal means 114 is received in the interior volume 110 before the thermal pack 100 is attached to a person or a piece of furniture, it will be appreciated that the thermal pack 100 can be attached to a person or a piece of furniture before the thermal means 114 is received in the interior volume 110.

The thermal pack 100 is capable of reducing the amount of heat a heat pack or hot/cold pack radiates to its surroundings, which means more heat from the heat pack or hot/cold pack can be provided to a user. The thermal pack 100 is also capable of reducing the amount of heat absorbed by a cool pack or hot/cold pack from its surroundings, which means the cool pack or hot/cold pack can provide more cooling to a user.

Referring to FIG. 4, which shows an example thermal means 114, the thermal means of this figure is a hot/cold pack filled with sodium acetate, which is capable of providing heating and cooling. It will be appreciated that other forms of heat packs, cold packs, or hot/cold packs may be used with the thermal pack 100 without detracting from the present invention.

It can be seen that the thermal pack 100 described above is in the general form of a burn bag, however, it will be appreciated that the temperature pack 100 may take other forms without detracting from the present invention.

Although the thermal pack 100 has been described above as having a strap 104, it will be appreciated that the thermal pack 100 does not require a strap 104. In the case where the thermal pack 100 does not have a strap 104, it will be appreciated that the thermal pack 100 could simply rest, or be held by a user, against the desired position on the user's body.

Although not a therapeutic device the thermal pack can also be used to assist in the treatment of minor muscular injuries, cramps, aches and pains.

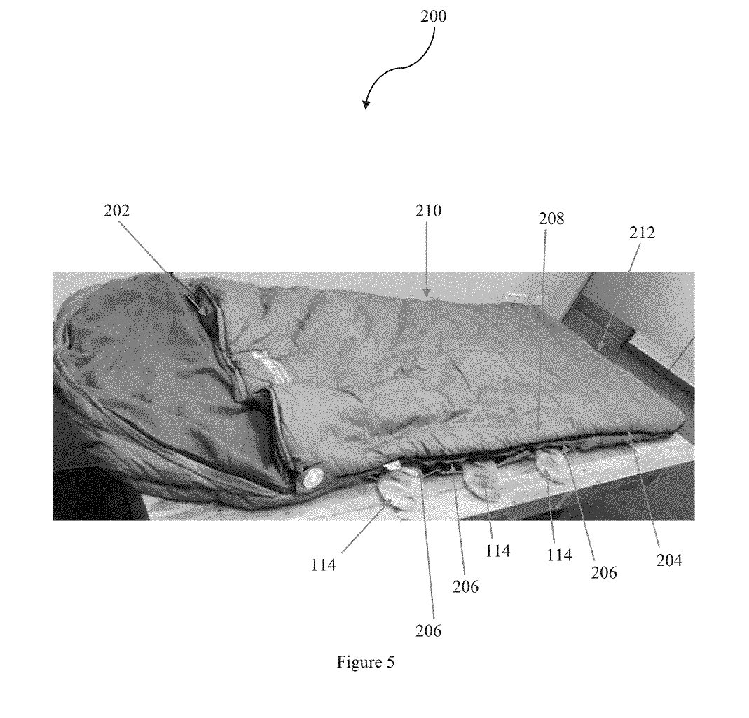

FIG. 5 shows a sleeping bag 200 according to another embodiment of the present invention. The sleeping bag 200 has a sleeping compartment 202 and a zip 204 to open and substantially close the sleeping compartment 202 to allow a user to get in and out of the sleeping compartment 202.

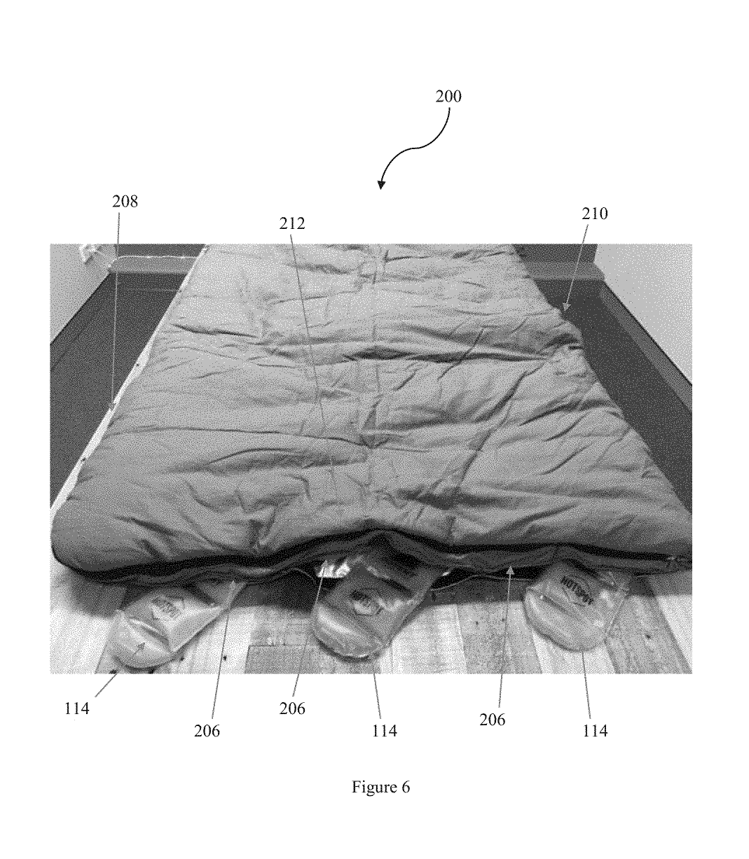

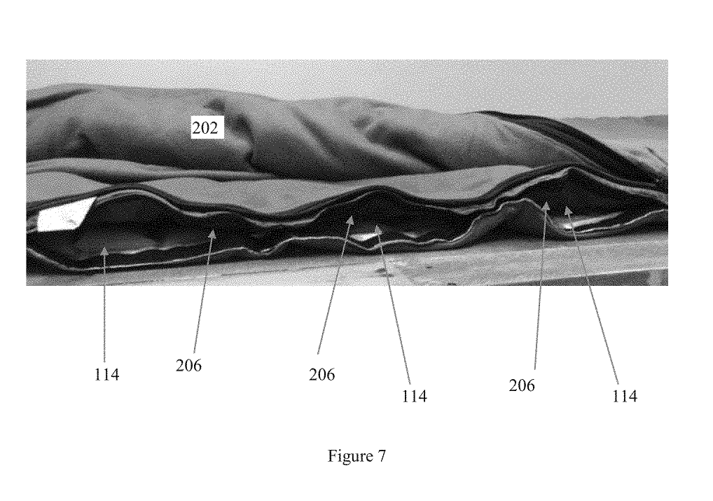

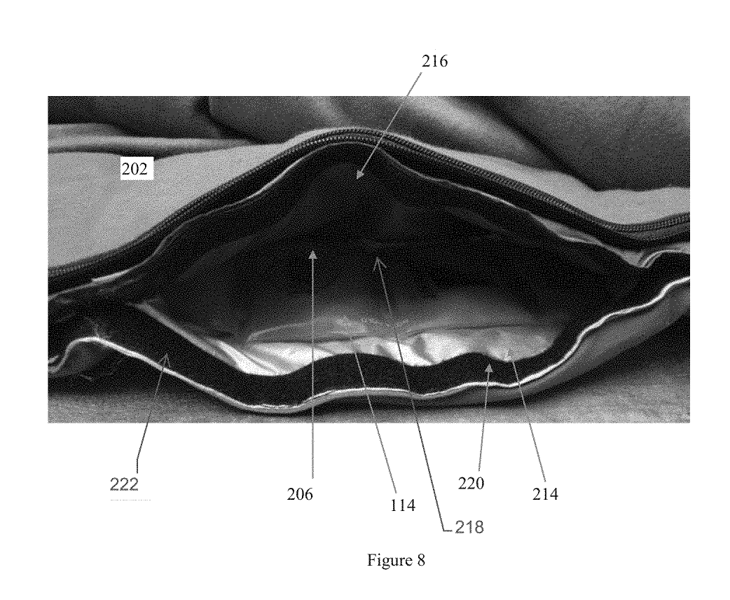

The sleeping bag 200 also has a number of adjacent thermal pockets 206 arranged along each longitudinal side 208 of the sleeping bag 200. Each of the thermal pockets 206 is disposed between the sleeping compartment 202 and an outer surface of the sleeping bag 200 (see FIG. 7). Although FIG. 5 only shows the sleeping bag 200 having thermal pockets 206 arranged on the longitudinal side 208, it is envisaged that the other longitudinal side 210 of the sleeping bag 200 also has a number of adjacent thermal pockets 206. Referring to FIG. 6, the sleeping bag 200 also has a number of thermal pockets 206 arranged along the base 212 of the sleeping bag 200. Referring to FIG. 8, each thermal pocket 206 is dimensioned to removably receive a thermal means 114 therein.

Referring to FIG. 8, each thermal pocket 206 has a first wall 214 and a second wall 216 coupled to the first wall 214 to form an interior volume 218 and an opening 220. The interior volume 218 is configured to retain a thermal means 114 therein. Referring to FIG. 8, the opening 220 has a hook and loop fastener 222 for opening and closing the opening 220. It will be appreciated that opening the opening 220 with the hook and loop fastener 222 allows a thermal means 114 to be received and removed from the interior volume 218, and that closing the opening 220 with the hook and loop fastener 222 allows a thermal means 114 to be retained in the interior volume 218. It is envisaged that the opening 220 can comprise any suitable means known in the art that is capable of allowing the opening 220 to be opened and closed.

Referring to FIG. 8, the first wall 214 has a thermal resistivity that is greater than a thermal resistivity of the second wall 216. Each thermal pocket 206 is disposed in the sleeping bag 200 such that the second wall 216 is adjacent the sleeping compartment 202 and the first wall 214 is adjacent the outer surface of the sleeping bag 200. It will be appreciated that the first wall 214 and the second wall 216 can be formed of any suitable material known in the art so long as the thermal resistivity of the first wall 214 is greater than the thermal resistivity of the second wall 216. As the thermal resistivity of the first wall 214 is greater than the thermal resistivity of the second wall 216, it will be appreciated that heat flow into, or out of, the interior volume 218 will be primarily through the second wall 216.

Use of the sleeping bag 200 for providing heat will now be described. A thermal means 114 configured to radiate heat (e.g. a heat pack or a hot/cold pack radiating heat) is received in the interior volume 218 of one or more of the thermal pockets 206 and the opening 220 closed using the hook and loop fastener 222.

Heat radiating from the thermal means 114 will primarily flow out of the interior volume 218 through the second wall 216, given that the thermal resistivity of the first wall 214 is greater than the thermal resistivity of the second wall 216.

As the second wall 216 is positioned adjacent the sleeping compartment 202, a majority of the heat radiating from the thermal means 114 will pass through the second wall 216 into the sleeping compartment 202. It will be appreciated that more heat will pass from the interior volume 218 to the sleeping compartment 202 compared to the amount of heat dissipated from the interior volume 218 through the first wall 214 and through the outer surface of the sleeping bag 200 and to the surroundings. As less heat is dissipated to the surroundings, more heat can be provided to the sleeping compartment 202 before the thermal means 114 reaches thermal equilibrium.

It will be appreciated that thermal means 114 disposed in one or more of the thermal pockets 206 of the sleeping bag 200 can be used to warm the sleeping compartment 202 before a person gets into the sleeping compartment 202.

Use of the sleeping bag 200 for providing cooling will now be described. A thermal means 114 configured to absorb heat (e.g. a cool pack or a hot/cold pack absorbing heat) is received in the interior volume 218 of one or more of the thermal pockets 206 and the opening 220 closed using the hook and loop fastener 222.

It will be appreciated that more heat will flow into the interior volume 218, and be absorbed by the thermal means 114, through the second wall 216 compared to the first wall 214, given the first wall 216 has a greater thermal resistivity than the thermal resistivity of the second wall 214.

As the second wall 216 is adjacent the sleeping compartment 202, the thermal means 114 will absorb heat from the sleeping compartment 202 through the second wall 218. Accordingly, it will be appreciated that the thermal means 114 will absorb less heat from the surroundings through the outer surface of the sleeping bag 200 and through the first wall 214 and more heat from the sleeping compartment through the second wall 216. As less heat is absorbed from the surroundings, more heat can be absorbed from the sleeping compartment 202 before the thermal means 114 reaches thermal equilibrium.

It will be appreciated that when camping, for example, a person may wish to cover their body to avoid being bitten by insects such as, for example, mosquitos. However, in warmer temperatures, sleeping in the sleeping compartment 202 may be too hot and uncomfortable. Use of thermal means 114 in the thermal pockets 206 to provide cooling may allow a person to comfortably sleep in the sleeping compartment 202 during warmer temperatures, which may provide some protection from being bitten by insects.

Although the invention has been described with reference to a specific example, it will be appreciated by those skilled in the art that the invention may be embodied in many other forms.

1. A thermal pack having a pouch, the pouch having:
a first wall; and
a second wall coupled to the first wall to define an interior volume, the interior volume configured to retain a thermal means in use,
wherein a thermal resistivity of the first wall is greater than a thermal resistivity of the second wall such that heat flow into, or out of, the interior volume is primarily through the second wall;
the thermal pack being configured for use with camping furniture or bedding.

2. The thermal pack of claim 1, wherein the first wall comprises a heat reflective material.

3. The thermal pack of claim 2, wherein the heat reflective material at least partially defines the interior volume.

4. The thermal pack of claim 1, wherein the interior volume is configured to removably receive the thermal means.

5. The thermal pack of claim 1, further comprising a means configured to removably attach the thermal pack to a person or a piece of furniture.

6. The thermal pack of claim 1, wherein the thermal pack is comprised within a sleeping bag, and wherein the second wall of the thermal pack is adjacent to a sleeping compartment of the sleeping bag.
